(19) United States
(12) Patent Application Publication
(10) Pub. No.: US 2026/0054057 A1
(43) Pub. Date: Feb. 26, 2026

(54) CARDIAC ELECTRODE ASSEMBLY WITH ANTI-MIGRATION FEATURE

(71) Applicant: Thabet Alsheikh, Pensacola, FL (US)

(72) Inventor: Thabet Alsheikh, Pensacola, FL (US)

(21) Appl. No.: 18/814,702

(22) Filed: Aug. 26, 2024

(51) Int. Cl. A61N 1/05

(52) U.S. Cl. CPC A61N 1/0573; A61N 2001/058

(57) ABSTRACT

A cardiac electrode assembly. The inventive electrode assembly includes an active fixation device, such as a helical electrode configured to screw into the myocardium or a vessel wall. The invention also includes an array of two or more reversed tines. The reverse tines bear against the heart wall or vessel wall and inhibit unwanted distal motion of the electrode assembly. Various deployment devices that aid in the installation of the inventive electrode assembly are also described.

### CROSS-REFERENCES TO RELATED APPLICATIONS

Not Applicable

### STATEMENT REGARDING FEDERALLY SPONSORED RESEARCH OR DEVELOPMENT

Not applicable

### MICROFICHE APPENDIX

Not applicable

### BACKGROUND OF THE INVENTION

### 1. Field of the Invention

This invention relates to the field of medical products. More specifically, the invention comprises a cardiac electrode assembly with active fixation that limits unwanted migration of the electrode assembly after it is installed.

### 2. Description of the Related Art

Intracardiac electrode assemblies are used in a variety of systems and procedures. Their use as a cardiac pacemaker electrode is particularly challenging since such installations often remain in an installed position for decades. FIG. 1 shows a prior art installation. Cardiac lead 26 is introduced through superior vena cava 12 (with a prior approach being typically made through the cephalic, axillary, or subclavian vein). The goal of the process is the affixation of an electrode within the right ventricle 16 apex 22. Lead 26 includes electrode assembly 24 on its distal end. A covering sheath or catheter is used to introduce and manipulate the lead through right atrium 14, through tricuspid valve 18, and into right ventricle 16. External manipulation device(s) 28 allow the physician to carefully guide the electrode assembly into position.

Electrode assembly 24 includes securing features that allow it to be attached to the endocardium. Once the electrode assembly is affixed, the placement sheath/catheter is withdrawn. The electrode assembly and lead remain in position. For a pacemaker installation, the lead will usually be connected to an implantable medical device.

There are many types of electrode assemblies available. These may be broadly classified as passive fixation leads and active fixation leads. FIG. 2 shows a passive fixation lead. Electrode assembly 24 includes conductor 36 covered by insulation 38. Tip electrode 30 is exposed on the device's distal end. The tip electrode is electrically connected to conductor 36. Displaced electrode 34 is often also provided. This allows the use of catheter navigation and mapping systems, such as described in my own U.S. Pat. No. 11,844,912. U.S. Pat. No. 11,844,912 is hereby incorporated by reference.

Electrode assembly 24 includes one or more tines 32 extending outward proximate tip electrode 30. These tines are intended to engage the trabeculations typically found on the interior heart wall. FIG. 4 shows the placement of the passive electrode assembly 24 with tip electrode 30 resting against the myocardium 46. Laterally extending tines 32 engage the trabeculae 44 present on the wall of the heart. The tines 34 act like barbs—limiting motion in the proximal direction (away from the wall of the heart).

The orientation of the tines with respect to the centerline of the lead assembly is important. Centerline axis 70 runs along the central axis of the lead assembly. A positive direction for centerline axis 70 is defined as the distal direction (toward or into the wall of the heart in the orientation shown in the view). Tines 32—when deployed—lie along an axis that is typically 135 degrees to 160 degrees angularly displaced from centerline axis 70.

The orientation of tines 32 with respect to the body of the lead is generally fixed. The tines remain in this state while the lead assembly is being advanced through the body prior to placement. The tines are small enough to be passed through the vasculature and the heart valves. Once placed as shown in FIG. 4, the tines remain with their fixed angle to the lead's central axis and their mechanical interference with the trabeculae holds the electrode assembly in place. However, the passive approach to electrode securement does sometimes fail. For this reason, lead/electrode assemblies having an active fixation mechanism have been developed and have become the most widely used design.

FIG. 3 shows an electrode assembly having active fixation. Electrode assembly 25 includes helical tip electrode 40 extending from its distal end. The helical tip electrode is made of conductive material. It is joined to conductor 36 at joint 42. Displaced electrode 34 is typically provided for an active fixation electrode assembly, as for the prior example.

FIG. 5 shows the installation of electrode assembly 25 in the myocardium. The physician advances electrode assembly 25 until its distal region 48 is proximate the desired installation site. The installation devices (catheter, etc.) are provided with a torque-transmitting engagement so that the physician can rotate electrode assembly 25—thereby screwing helical tip electrode into myocardium 46.

The helical engagement of the tip electrode 40 with the myocardium actively secures the electrode assembly to the wall of the heart. It may on some occasions, however, produce an unwanted result. The heart continues beating during and after the installation of electrode assembly 25. The heart beats approximately 100,000 times per day—and a pacemaker electrode may remain installed for one to two decades. Thus, the electrode assembly is expected to endure over 300 million contractions of the heart. In some instances the presence of helical tip electrode 40 causes the electrode assembly to advance further into the myocardium than is desirable. In fact the helical tip electrode may eventually migrate all the way through the wall of the heart.

The present invention provides a new type of electrode assembly that retains the benefits of active fixation while minimizing the possibility of unwanted migration.

### BRIEF SUMMARY OF THE PRESENT INVENTION

The present invention comprises a cardiac electrode assembly. The inventive electrode assembly includes an active fixation device, such as a helical electrode configured to screw into the myocardium or a vessel wall. The invention also includes an array of two or more reversed tines. The reversed tines bear against the heart wall or vessel wall and inhibit unwanted distal motion of the electrode assembly. Various deployment devices that aid in the installation of the inventive electrode assembly are also described.

### BRIEF DESCRIPTION OF THE SEVERAL VIEWS OF THE DRAWINGS

FIG. 1 is an elevation view with a partial section, showing the installation of a prior art electrode assembly in a human heart.

FIG. 2 is an elevation view with a partial section, showing a prior art passive fixation electrode assembly.

FIG. 3 is an elevation view with a partial section, showing a prior art active fixation electrode assembly.

FIG. 4 is an elevation view, showing the installation of the electrode assembly of FIG. 2 in a human heart.

FIG. 5 is an elevation view, showing the installation of the electrode assembly of FIG. 3 in a human heart.

FIG. 6 is an elevation view, showing an embodiment of the present invention.

FIG. 7 is an elevation view, showing the installation of the electrode assembly of FIG. 6 in a human heart.

FIG. 8A is an elevation view with a partial section, showing the use of an installation catheter with the inventive electrode assembly.

FIG. 8B is a perspective view, showing the inventive electrode assembly contained within an installation catheter.

FIG. 9 is an elevation view with a partial section, showing the installation of an embodiment of the inventive electrode assembly in a human heart.

FIG. 10 is a section view, showing the installation of an embodiment of the inventive electrode assembly in a human heart.

FIG. 11 is an elevation view with a partial section, showing the installation of an embodiment of the inventive electrode assembly in a human heart.

FIG. 12 is an elevation view with a partial section, showing the installation of an embodiment of the inventive electrode assembly in a human heart.

### REFERENCE NUMERALS IN THE DRAWINGS

10 heart
12 superior vena cava
14 right atrium
16 right ventricle
18 tricuspid valve
20 septum
22 apex
24 electrode assembly
25 electrode assembly
26 lead
28 external manipulation device
30 tip electrode
32 tine
34 displaced electrode
36 conductor
38 insulation
40 helical tip electrode
42 joint
44 trabeculae
46 myocardium
48 distal region
50 reversed tine
52 surface
54 installation catheter
56 tip region
58 lateral slot
60 body
62 electrode assembly
64 knuckle
70 centerline axis

### DETAILED DESCRIPTION OF THE INVENTION

The following descriptions pertain to embodiments suitable for use as pacemaker leads. However, as those skilled in the art will appreciate, the invention can be used for many different applications and is by no means limited to the field of pacemakers. The scope of the invention should thus be set by the claims presented rather than the exemplary embodiments.

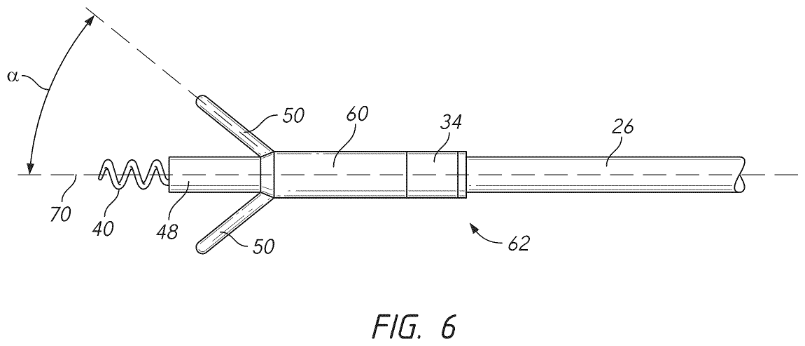

FIG. 6 provides an elevation view of an inventive lead/electrode assembly 62 made according to the present invention. Lead 26—including a conductor within a surrounding insulation layer—is provided as for the prior art. The lead attaches to body 60. Displaced electrode 34 is optionally provided for use with catheter navigation systems. An active fixation device is provided on distal region 48. In this example, helical tip electrode 40 extends in the distal direction from distal region 48.

Helical tip electrode 40 can be screwed into the myocardium by rotating body 60. The helical tip electrode is electrically connected to the conductor within lead 26. Thus, the helical tip electrode creates an electrical path from the conductor within the lead to the myocardium.

Body 60 also includes a plurality of reversed tines 50 extending laterally outward. The orientation of the reversed tines is significant. Each tine extends laterally and distally away from the body, so that the tip of each reversed tine is displaced in the distal direction from its root. Centerline axis 70 runs down the middle of body 60. The centerline of each reversed tine is offset an angle α from centerline axis 70. The angle α is preferably in the range of 20 degrees to 70 degrees and even more preferably in the range of 20 degrees to 50 degrees.

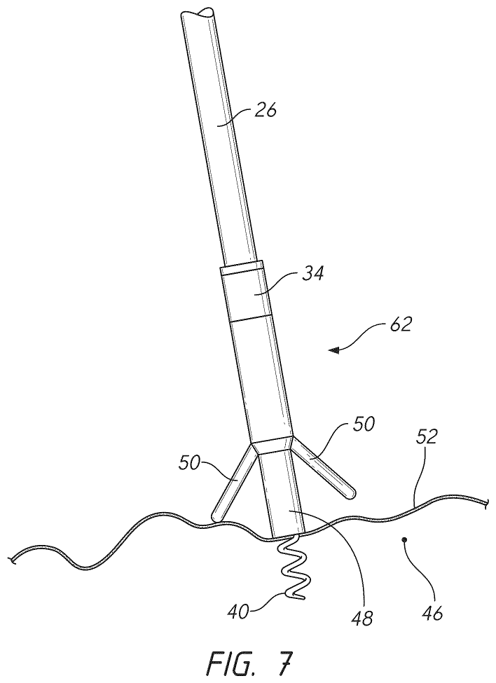

FIG. 7 shows the installation of the inventive electrode assembly 62 in myocardium 46. Helical tip lead 40 is screwed into myocardium 46 by rotating body 60. The installation devices (such as an encompassing catheter or sheath) are generally used to apply the rotational torque during the installation process—though the invention is not limited to this method. The reader will observe how the distal tips of the reversed tines 50 contact surface 52 as distal region 48 reaches the surface of the myocardium. Reversed tines 50 resist further penetration of the electrode assembly into the myocardium.

The reader should bear in mind that an installation such as shown in FIG. 7 is intended to remain affixed for many years (10 to 20 years in some cases). The myocardium shown is of course not stationary. It averages over 1 contraction per second—accumulating over 300 million contractions per year. This constant motion can produce unwanted additional penetration of the electrode assembly into the heart. Reversed tines 50 resist this unwanted migration by providing a non-penetrating but positive contact with surface 52.

Reversed tines 50 must be made of bio-compatible material. It is preferable for reversed tines 50 to also be made of a soft and pliable material. In the example shown the distal tips are smooth and rounded in order to avoid any irritation of the heart wall as the heart beats.

It is also possible to make the reversed tines from a soft and electrically conductive material (or provide a soft material with an electrically-conductive outer layer). The provision of an electrically conductive portion for some or all of the outer surfaces of the reversed tines allows them to serve as an electrode for cardiac pacing or for stimulation of nerves adjacent to blood vessels when the lead/electrode assembly is actively fixated within the vein wall in areas close to the target nerve.

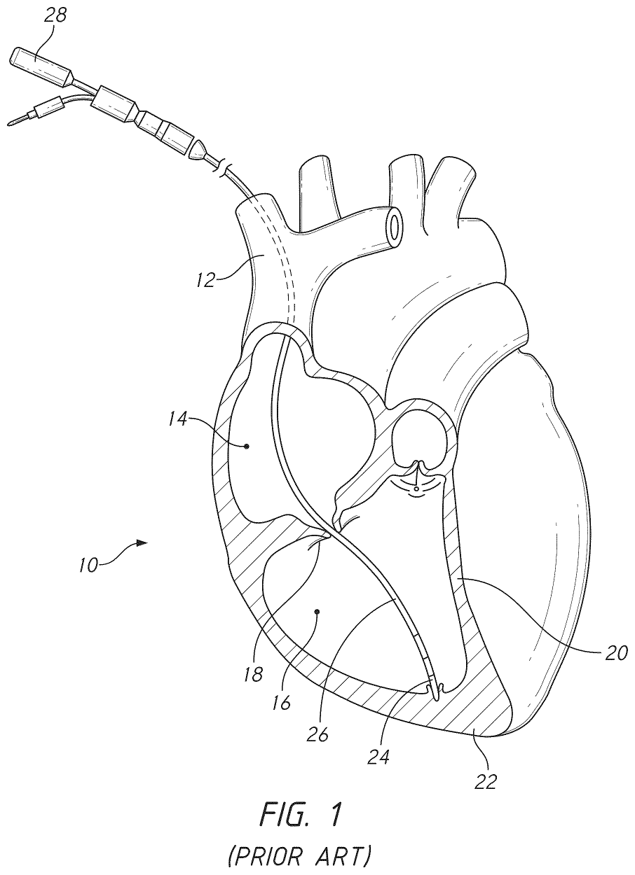

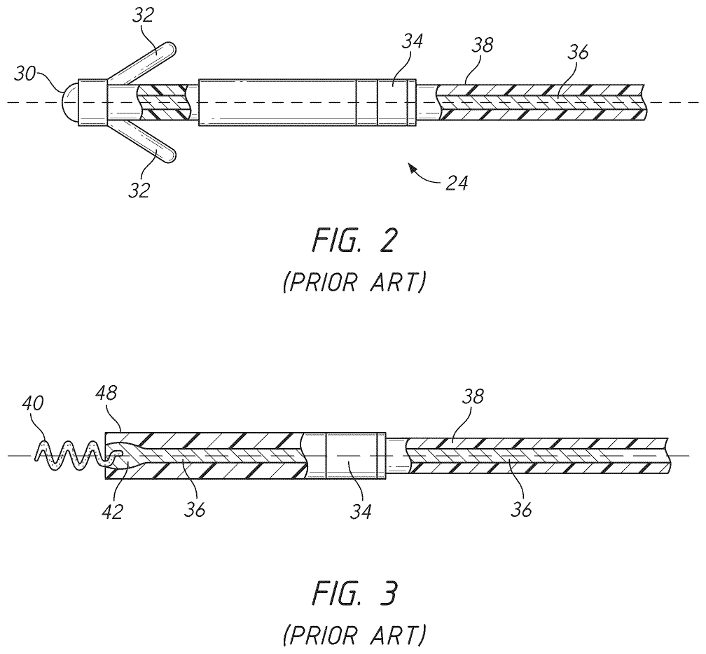

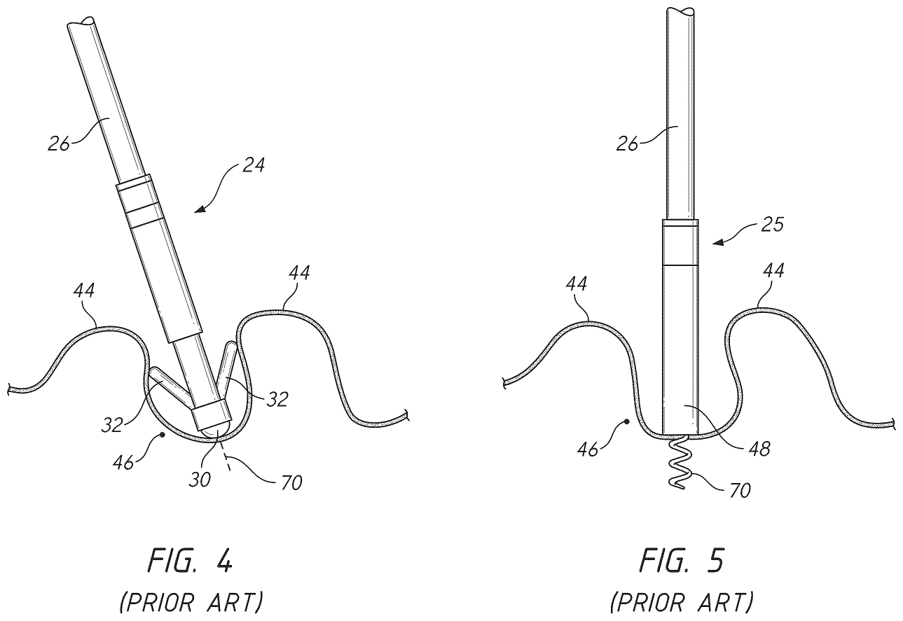

The reversed tines are preferably made small enough so that they can be introduced through the vasculature and valves in the same configuration as when they are attached to the patient's anatomy. The tines are small enough that introduction in a deployed state is feasible, as for the prior art conventional tines. However, in some instances, the geometry of the reversed tines may cause problems—such as for the process of introducing the lead and feeding it into the heart (see FIG. 1). For these situations, it is possible (but not required) to provide both a “stowed” position and a “deployed” position for the inventive electrode assembly. In the stowed position reversed tines 50 are urged inward toward body 60. In the deployed position they extend outward as shown in FIG. 6.

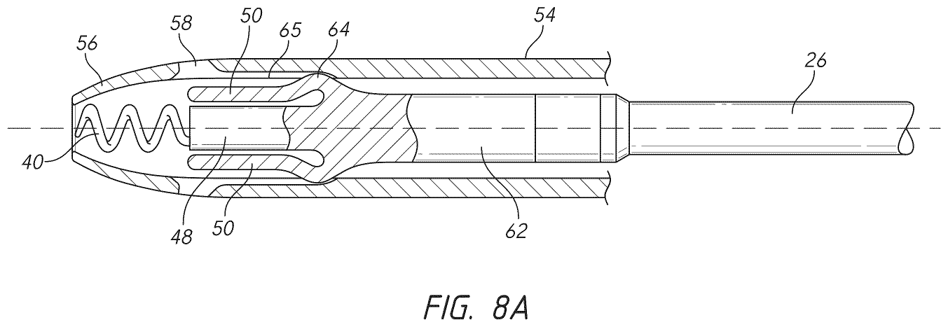

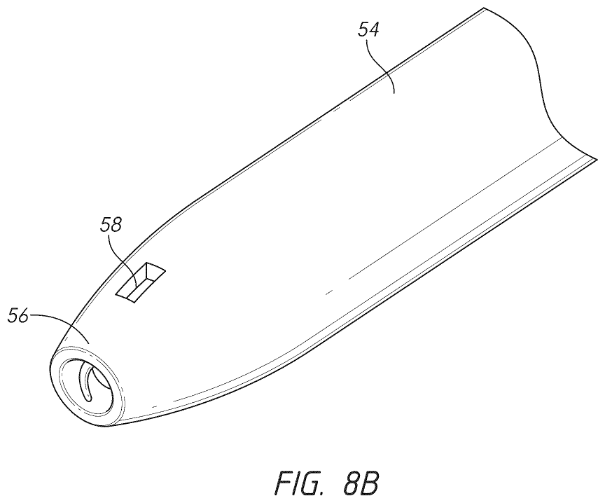

FIG. 8A shows an embodiment for the reversed tines in which a stowed position is provided. This alternate embodiment of the inventive electrode assembly is enclosed within an installation catheter 54. Installation catheter 54 slides over the exterior of lead 26 and electrode assembly 62. In the example shown, the installation catheter urges reversed tines 50 inward against distal region 48. Each reversed tine 50 is joined to body 60 via a curved knuckle 64. This knuckle protrudes radially outward. The knuckle bears against the inner surface of the installation catheter, providing a frictional force that tends to keep the installation catheter and the electrode assembly engaged. Each knuckle also rides in a corresponding groove 65 in the interior surface of the installation catheter. The interface between each knuckle 64 and its corresponding groove 65 prevents rotation of the electrode assembly 62 with respect to the catheter 54.

Tip region 56 of the installation catheter preferably closes over helical tip electrode 40 so that the tip electrode does not contact any soft structures while the electrode and lead are being introduced through the superior vena cava and through the heart. FIG. 8B provides a perspective view of the same assembly in the vicinity of tip region 56. The smooth exterior provides for an easier passage into the desired location in the heart. Multiple 58 are provided in the installation catheter—with each slot being aligned with one of the reversed tines. The installation catheter may provide many other features—such as wire helices used for manipulation and external electrodes used in an external navigation system.

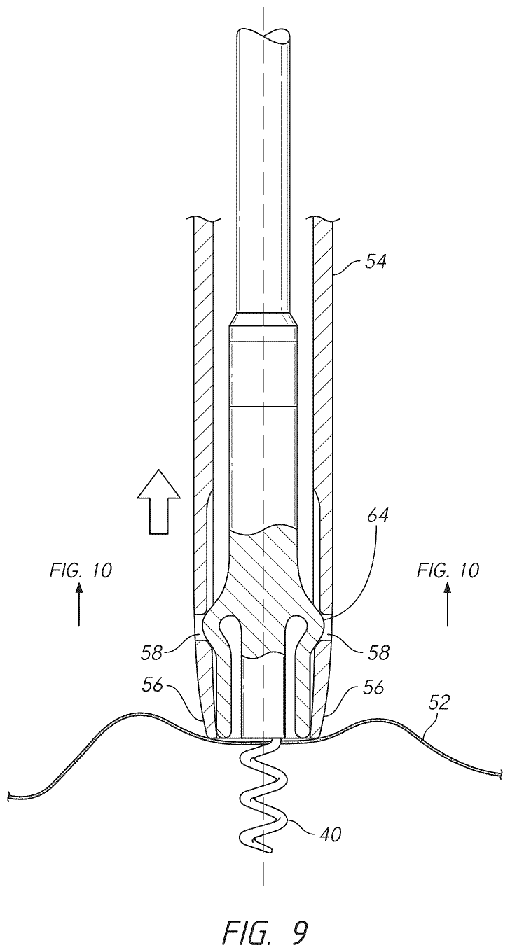

FIGS. 9-12 illustrate an exemplary installation process. The assembly of FIG. 8A is advanced until tip region 56 lies proximate the desired installation site. As then shown in FIG. 9, installation catheter 54 is withdrawn a short distance in the proximal direction as indicated by the arrow. As the catheter is withdrawn, each knuckle 64 slides along within its corresponding groove 65 until it pops out into its corresponding lateral slot 58. This serves as a “detent” that secures the installation catheter in the partially withdrawn position shown in FIG. 9.

Tip region 56 of catheter 54 is made of flexible material so that it can expand outward as shown. The flexibility can be obtained by providing a thinner wall section for the catheter in the area of tip region 56. Alternatively, tip region 56 may made as a separate piece that is joined to the balance of the catheter. In this approach the tip region can be made using a more flexible material than the balance of the catheter.

In the configuration shown in FIG. 9, helical tip electrode 40 is fully exposed but the reversed tines are still urged inward. The assembly is ready to be installed in the wall of the heart. The physician applies torque to rotate the electrode body and screw the helical tip electrode 40 into the myocardium as shown (In other embodiments the helix is screwed into the myocardium by rotating only the inner conductor and not by rotating the whole lead body). For an embodiment where the entire lead body is rotated the reversed tines do not strike adjoining structures since they are still in the stowed configuration. The torque for this embodiment is transmitted from the installation catheter 54 to the electrode assembly (via knuckles 64 being secured within lateral slots 58). The installation catheter extends to the exterior of the body. Manual manipulation devices can be provided on the external portions of the catheter. The physician grips these features and rotates the assembly as desired. Fluoroscopy may be used to ensure a proper installation.

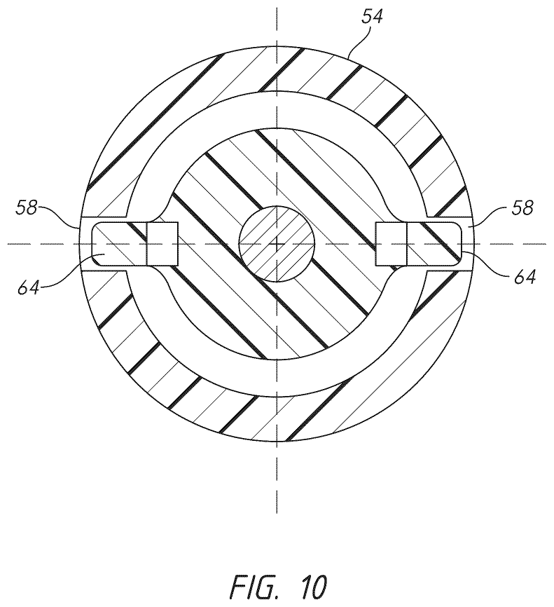

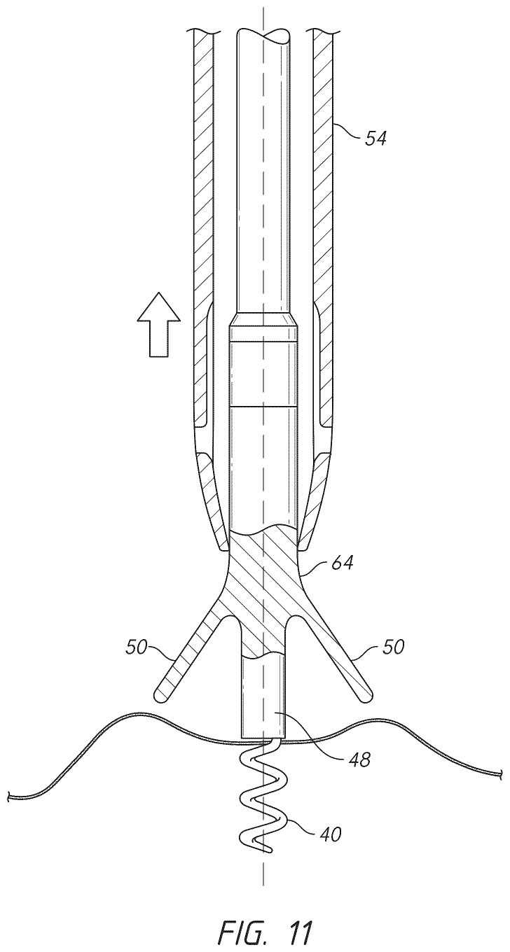

FIG. 10 provides a sectional view looking in the proximal direction through the assembly of FIG. 9. The reader will note the positive engagement between knuckles 64 and the lateral channels 58. This positive engagement allows the desired torque transfer between the installation catheter 54 and the electrode assembly.

Once the helical tip electrode is screwed into position, the physician further withdraws the installation catheter as shown in FIG. 11. Once the tip region of the catheter slides in the proximal direction over knuckles 64, reversed tines 50 pop outward into the deployed position shown. They will then remain in the deployed position for the life of the installation.

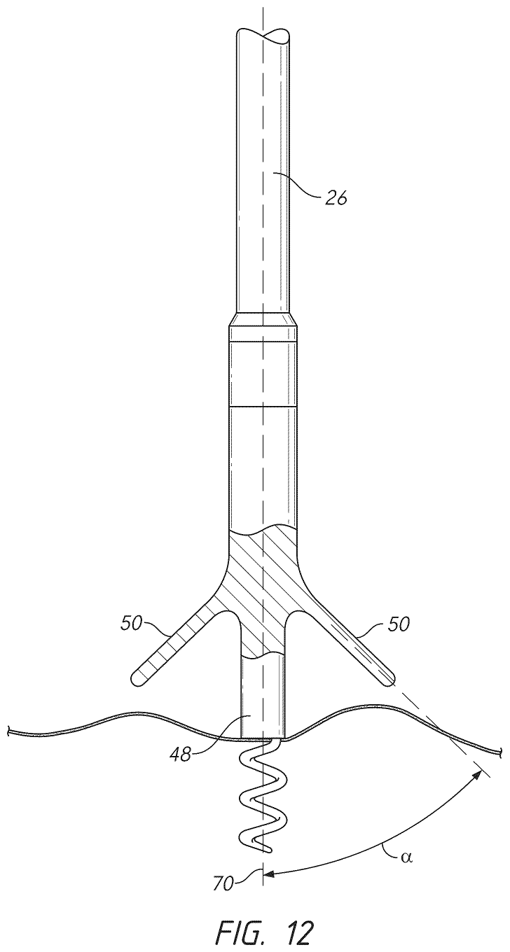

FIG. 12 shows again the angle α between centerline axis 70 and an axis lying along the center of each reverse tine 50. As stated previously, the angle α is preferably in the range of 20 degrees to 70 degrees and even more preferably in the range of 20 degrees to 50 degrees. The reader will note how the angle has increased between the state shown in FIG. 11 and the state shown in FIG. 12. Where a molded polymer is used, the initial deployment angle will increase gradually until reaching a stable position. FIG. 12 represents a final stable position for this embodiment.

The use of the reference centerline axis 70 does not mean that the electrode assembly and other structures must be radially symmetric—though many examples are in fact radially symmetric. The use of two reversed tines in the example should likewise not be viewed as a limitation. Many other configurations of reversed tines are possible.

Many other features can be added to the inventive embodiments shown. These features may be added singly or in combinations. These features include:
1. An embodiment with three, four, or more reversed tines;
2. An embodiment with enlarged contact pads on the distal ends of the reversed tines;
3. An embodiment where the reversed tines are made of a flexible polymer;
5. An embodiment where the reversed tines are made of metal;
6. An embodiment where the reversed tines include a metal core surrounded by a flexible polymer;
7. An embodiment wherein a mechanical hinge mechanism is used to connect the reversed tines to the balance of the electrode assembly;
8. An embodiment where some or all of the reversed tines are conductive; and
9. An embodiment where some portion of each reversed tine is conductive.

Although the preceding description contains significant detail, it should not be construed as limiting the scope of the invention but rather as providing illustrations of the preferred embodiments of the invention. Those skilled in the art will be able to devise many other embodiments that carry out the present invention. Thus, the language used in the claims shall define the invention rather than the specific embodiments provided.

Having described my invention, I claim:

1. A cardiac electrode assembly, comprising:
(a) a body having a centerline axis and a distal region;
(b) a conductor passing through said body;
(c) a helical tip electrode extending from said distal region in a distal direction;
(d) a plurality of reversed tines extending laterally outward from said body; and
(e) wherein each reversed tine lies along an axis forming an angle between 20 degrees and 70 degrees from said centerline axis.

2. The cardiac electrode assembly as recited in claim 1, further comprising a displaced electrode on said body.

3. The cardiac electrode assembly as recited in claim 1, further comprising:
(a) a sleeve passed over said body and said plurality of reversed tines; and
(b) wherein said sleeve urges each of said reversed tines inward toward said centerline axis.

4. The cardiac electrode assembly as recited in claim 3:
(a) wherein said sleeve comprises a tip region; and
(b) wherein said sleeve comprises a plurality of channels proximate said tip region, with each of said channels being sized to receive one of said reversed tines.

5. The cardiac electrode assembly as recited in claim 4, wherein said plurality of channels is configured to transmit torque to said reversed tines, thereby transmitting torque from said sleeve to said body.

6. The cardiac electrode assembly as recited in claim 1, wherein said reversed tines are made of resilient material capable of bending in against said distal region without fracturing.

7. The cardiac electrode assembly as recited in claim 3, wherein said reversed tines are made of resilient material capable of bending in against said distal region without fracturing.

8. A cardiac electrode assembly, comprising:
(a) a body having a distal region;
(b) a conductor passing through said body;
(c) a helical tip electrode extending from said distal region in a distal direction;
(d) a plurality of reversed tines extending from said body; and
(e) wherein each reversed tine lies extends both laterally outward from said body and in a distal direction.

9. The cardiac electrode assembly as recited in claim 8, further comprising a displaced electrode on said body.

10. The cardiac electrode assembly as recited in claim 8, further comprising:
(a) a sleeve passed over said body and said plurality of reversed tines; and
(b) wherein said sleeve urges each of said reversed tines inward toward a centerline axis of said body.

11. The cardiac electrode assembly as recited in claim 10:
(a) wherein said sleeve comprises a tip region; and
(b) wherein said sleeve comprises a plurality of channels proximate said tip region, with each of said channels being sized to receive one of said reversed tines.

12. The cardiac electrode assembly as recited in claim 11, wherein said plurality of channels is configured to transmit torque to said reversed tines, thereby transmitting torque from said sleeve to said body.

13. The cardiac electrode assembly as recited in claim 8, wherein said reversed tines are made of resilient material capable of bending in against said distal region without fracturing.

14. The cardiac electrode assembly as recited in claim 10, wherein said reversed tines are made of resilient material capable of bending in against said distal region without fracturing.
